(19) United States
(12) Patent Application Publication
(10) Pub. No.: US 2012/0136152 A1
(43) Pub. Date: May 31, 2012

(54) EFFICIENT SYNTHETIC METHOD OF 18F-MEFWAY PRECURSOR

(75) Inventors: Young Hoon RYU, Seoul (KR); Tae Joo JEON, Seoul (KR); Chul Hoon KIM, Gyeonggi-do (KR); Jae Yong CHOI, Seoul (KR)

(73) Assignee: INDUSTRY-ACADEMIC COOPERATION FOUNDATION, YONSEI UNIVERSITY, SEOUL (KR)

(21) Appl. No.: 12/954,680

(22) Filed: Nov. 26, 2010

(51) Int. Cl. C07D 401/12

(52) U.S. Cl. 544/360

(57) ABSTRACT

The present invention relates a novel method for preparing an ¹⁸F-mefway precursor. The present invention provides an efficient synthetic method of an ¹⁸F-mefway precursor, which comprises an improved the acid chloride coupling reaction and proper reduction condition to suppress breakdown of amide bond and can obtain the precursor in high yield.

### TECHNICAL FIELD

The present invention relates a novel method for preparing an ¹⁸F-mefway precursor.

### BACKGROUND ART

The serotonin (5-HT) system, one of the most important neurotransmitter systems, has been classified into seven subtypes (5-HT₁₋₇). Among these subfamilies, 5-HT_{1A} receptors in the central nervous system are strongly implicated in psychiatric disorders such as depression, anxiety and schizophrenia. Thus, molecular imaging agents for the 5-HT_{1A} have been intensively studied since the past decade. Positron emission tomography (PET) as a non-invasive imaging technique with a high-sensitivity (10⁻⁹˜10⁻¹² M) and quantitative property provides the means to visualize receptor densities in living system. PET can play an important role both in assessing the neuropsychiatric disorders and in therapies with already developed pharmaceuticals. It is known that only high affinity agonists bind to receptors while antagonists' binding is relatively insensitive to their affinity. Thereby, a number of antagonist tracers have been developed as PET imaging agents on the basis of the WAY-100635 which is a selective antagonist for 5-HT_{1A} receptors at both somatodendritic and postsynaptic receptor sites (Fletcher, A. et al., Br. J. Pharmacol. 1993, 112, 91; Lang, L. et al., J. Med. Chem. 1999, 42, 1576-1586; Pike, V. W. et al., Nucl. Med. Biol. 2000, 27, 449-455; Lang, L. et al., Bioorg. Med. Chem. 2006, 14, 3737-3748; Marchais, S. et al., Bioorg. Med. Chem. 2001, 9, 695-702).

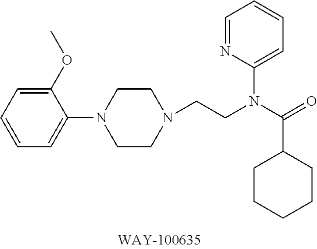

Structural analogues were designed to have stability to metabolism with high affinity, and selectivity for the 5-HT_{1A}. The most important drawback of these compounds is the significant defluorination that causes low-quality images due to contamination of ¹⁸F-fluoride ion in the skull. Recently, Neil Saigal et al. reported ¹⁸F-Mefway ((N-2-{2-[4-(2-methoxyphenyl)piperazinyl]ethyl}-N-(2-pyridyl)-N-(4-¹⁸F-fluoro-methylcyclo-hexane)carboxamide) as a potent PET agent for 5-HT_{1A} receptors.

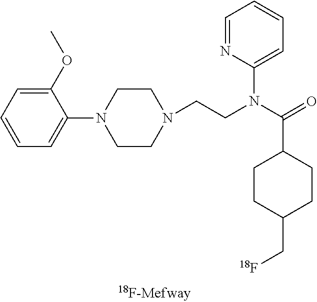

This compound had not only high target-to-non target ratios in receptor rich regions but also little defluorination (Saigal, N. et al., J. Nucl. Med. 2006, 47, 1697-1706). However, synthesis of its precursor, trans-N-{2-[4-(2-methoxyphenyl)piperazinyl]ethyl}-N-(2-pyridyl)-N-(4-tosyloxymethylcyclohexane)carboxamide, prior to the fluorination was quite inefficient with the low overall yield for the development of radiopharmaceutical due to the significant breakdown of amide bond, i.e., up to 70% of starting substrate, during the reduction of carbomethoxy group toward WAY-100635 derivative.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention provides a novel method for preparing an ¹⁸F-mefway precursor. The present invention provides an efficient synthetic method of an ¹⁸F-mefway precursor, which comprises an improved the acid chloride coupling reaction and proper reduction condition to suppress breakdown of amide bond and can obtain the precursor in high yield.

In one aspect of the inventive subject matter, a process for preparing a compound of the formula 6

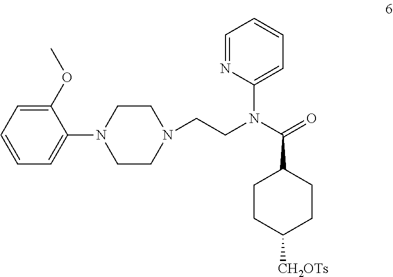

comprises the steps of:

a) adding oxalyl chloride to 4-carbomethoxycyclohexane-1-carboxylic acid to form a reaction mixture and reacting the reaction mixture with a compound of the formula 3

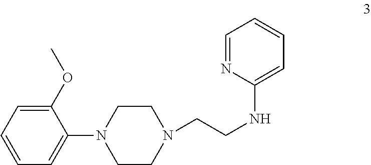

to form a compound of the formula 4

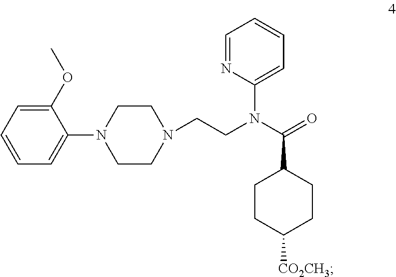

b) reduction of the compound of the formula 4 with LiAlH₄ in diethyl ether to form a compound of the formula 5

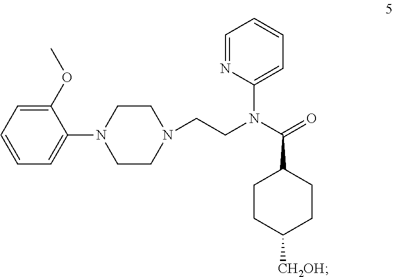

and

c) tosylating the compound of the formula 5 with p-toluenesulfonyl anhydride or p-toluenesulfonyl chloride to form the compound of the formula 6.

### DETAILED DESCRIPTION OF THE DISCLOSURE

The inventors have discovered an efficient synthetic method of an ¹⁸F-mefway precursor.

In one aspect of the inventive subject matter, a process for preparing a compound of the formula 6

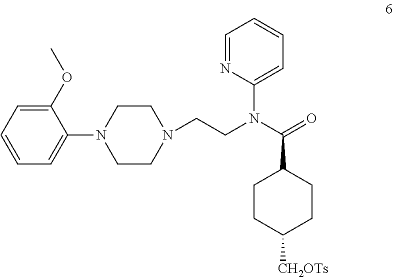

comprises the steps of:

a) adding oxalyl chloride to 4-carbomethoxycyclohexane-1-carboxylic acid to form a reaction mixture and reacting the reaction mixture with a compound of the formula 3

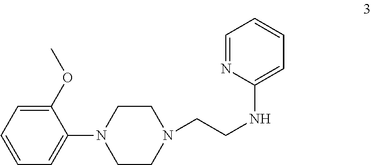

to form a compound of the formula 4

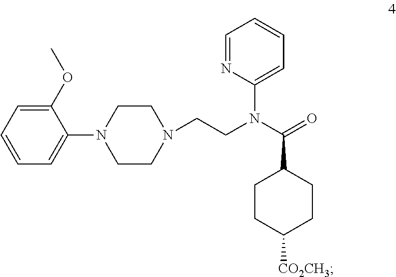

b) reduction of the compound of the formula 4 with LiAlH₄ in diethyl ether to form a compound of the formula 5

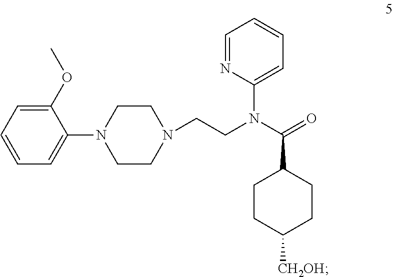

and

c) tosylating the compound of the formula 5 with p-toluenesulfonyl anhydride or p-toluenesulfonyl chloride to form the compound of the formula 6.

Preferred process of adding oxalyl chloride to 4-carbomethoxycyclohexane-1-carboxylic acid, but are not limited to the following, is in CH₂Cl₂.

Preferred process further comprises the step of reacting 2-aminopyridine with chloroacetyl chloride and then adding a reducing agent to form the compound of the formula 3. The reducing agent, but are not limited to the following, is LiAlH₄, NaBH₄, Ca(BH₄)₂, diisobutylaluminumhydride, AlH₃ or sodium bis(2-methoxy-ethoxy)aluminum hydride.

In one aspect of the inventive subject matter, a process for preparing a compound of the formula 5

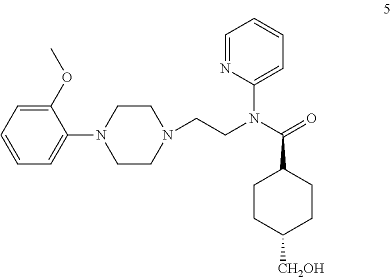

comprises the steps of:

a) adding oxalyl chloride to 4-carbomethoxycyclohexane-1-carboxylic acid to form a reaction mixture and reacting the reaction mixture with a compound of the formula 3

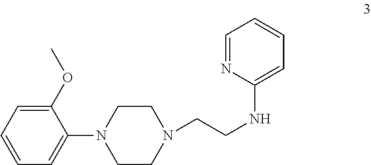

to form a compound of the formula 4

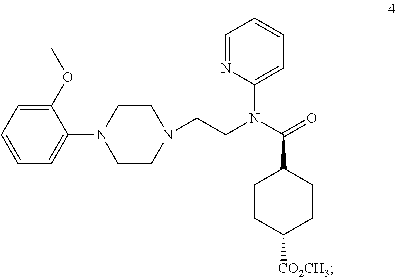

and

b) reduction of the compound of the formula 4 with LiAlH₄ in diethyl ether to form the compound of the formula 5.

In one aspect of the inventive subject matter, a process for preparing ¹⁸F-mefway, which process comprises the steps of:

a) adding oxalyl chloride to 4-carbomethoxycyclohexane-1-carboxylic acid to form a reaction mixture and reacting the reaction mixture with a compound of the formula 3

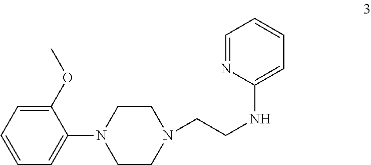

to form a compound of the formula 4

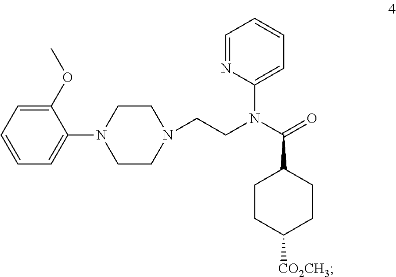

b) reduction of the compound of the formula 4 with LiAlH₄ in diethyl ether to form a compound of the formula 5

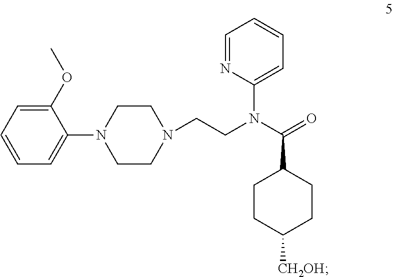

c) tosylating the compound of the formula 5 with p-toluenesulfonyl anhydride or p-toluenesulfonyl chloride to form a compound of the formula 6

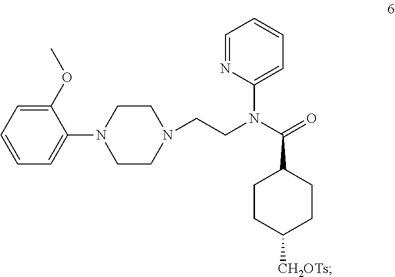

and

d) reacting the compound of the formula 6 with ¹⁸F-fluoride to form ¹⁸F-mefway.

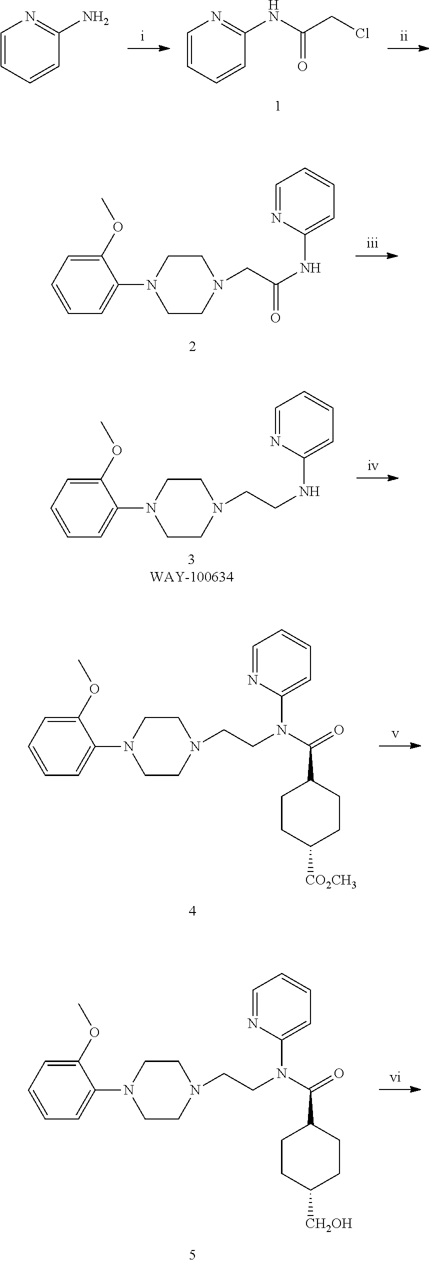

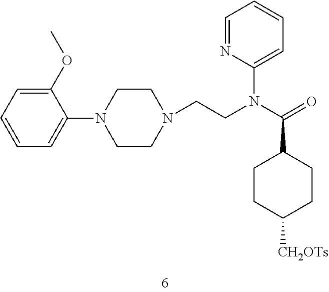

WAY-100634 (3) was prepared as previously described. (Pike, V. W. et al., Med. Chem. Res. 1995, 5, 208-277) Reaction of 2-aminopyridine with chloroacetyl chloride at room temperature provided the 2-(chloroacetyl)amidopyridine (1) as a intermediate. The treatment of the intermediate with 1-(2-methoxyphenyl)piperzines in DMF at 80° C. in the presence of K₂CO₃ and NaI gave the corresponding N-2-[2-{4-(2-methoxyphenyl)-1-piperazinyl}ethyl]amidopyridine (2), which is subsequently reduced to desired product (3), using LiAlH₄ in THF at room temperature. The WAY-100634 was coupled to trans-4-carbomethoxycyclohexanecarbonyl chloride in dichloromethane in the presence of triethylamine at room temperature, yielding trans-N-2-{2-[4-(2-methoxy-phenyl)piperazinyl]ethyl}-N-(2-pyridyl)-N-(4-carboxymethylcyclohexane) carboxamide (4). In this reaction, the inventors used oxalyl chloride as a coupling agent instead of benzotriazole-1-yl-oxy-tris-(dimethylamino)-phosphonium hexafluorophosphate (BOP) to enhance reactivity of carboxylic acid. This method improved the reaction yield up to 83% higher than previously reported method using BOP. Specific reduction of carbomethoxy group in (4) was conducted in LiAlH₄ in Et₂O at 0° C. to provide trans-N-2-{2-[4-(2-methoxyphenyl)piperazinyl]ethyl}-N-(2-pyridyl)-N-(4-hydroxymethylcyclohexane)carbox-amide (5) in a 57% yield. This new procedure improves the reaction yield significantly compared to the previous protocol. (Saigal, N. et al., J. Nucl. Med. 2006, 47, 1697-1706)

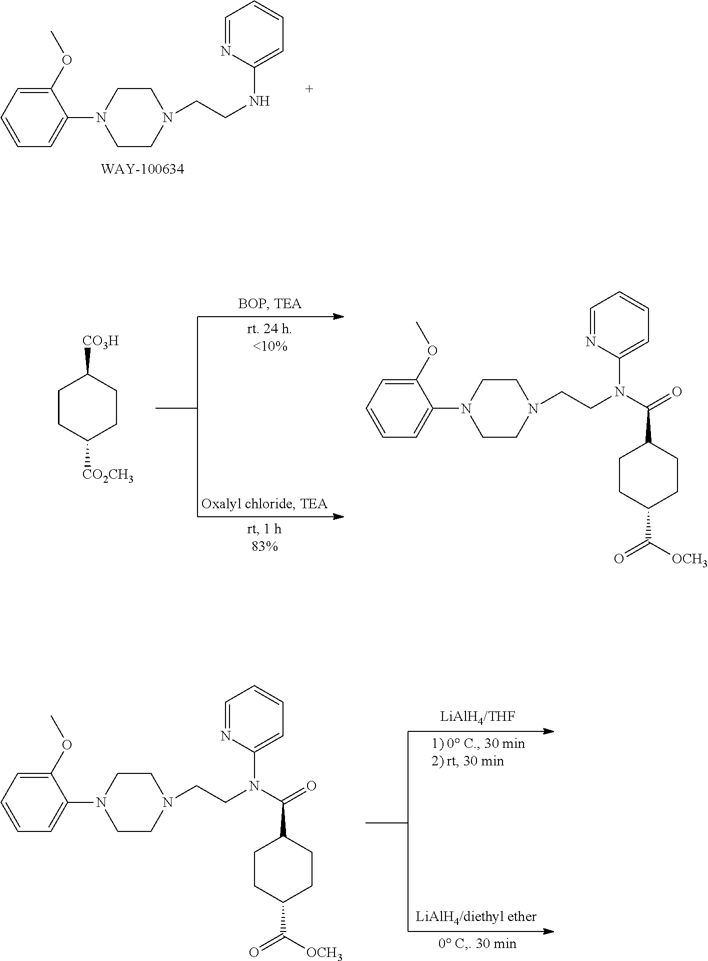

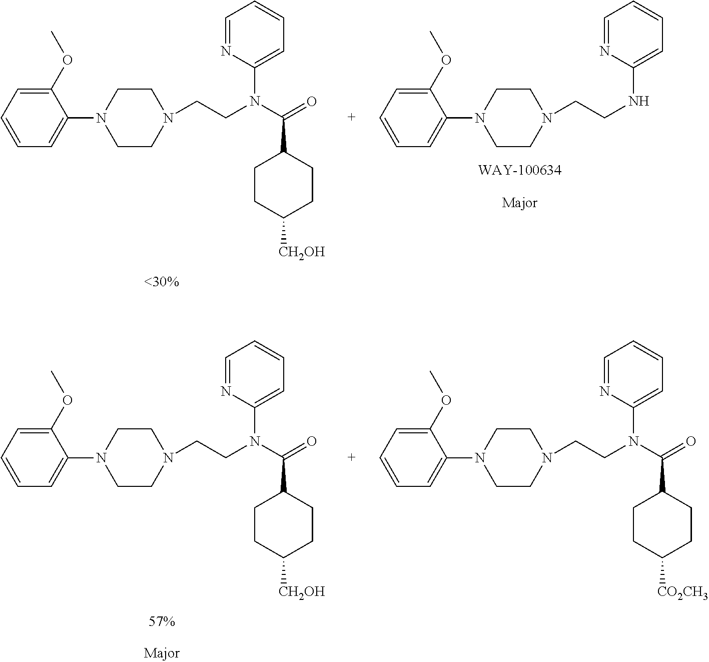

The treatment of same substrate (5) with LiAlH₄ in THF facilitated significant breakdown of the amide bond to yield compound 3 in more than 60% yield due to the possible presence of adventitious water in THF or unusual reactivity of LiAlH₄ in THF solution. For the preparation of the tosylated Mefway precursor (6), the hydroxyl compound (5) was reacted with p-toluenesulfonyl anhydride or p-toluenesulfonyl chloride at room temperature in dichloromethane in the presence of triethylamine to afford the precursor of ¹⁸F-Mefway in 75% or 60% yield.

### EXAMPLES AND COMPARATIVE EXAMPLES

### Example 1

### Preparation of N-2-(2-Chloroethyl)amidopyridine (1)

Chloroacetyl chloride (2.39 mL, 32.97 mmol) was slowly added to the mixture of 2-aminopyridine (1.88 g, 19.98 mmol) and TEA (4.30 mL, 30.69 mmol) in dry dichlorometane (100 mL) at 0° C. The reaction mixture was stirred at room temperature under an Ar atmosphere for 3 h. The organic layer was extracted with CH₂Cl₂, washed with water, and dried over anhydrous MgSO₄. The residue was purified by flash column chromatography (3:1 hexane/ethyl acetate) to give product (2.68 g, 79%) as a white solid.

¹H NMR (CDCl₃, 300 MHz) δ 4.20 (s, 2H), 7.09-7.14 (m, 1H), 7.77 (m, 1H), 8.22-8.28 (m, 2H), 9.45 (s, 1H); ¹³C NMR (CDCl₃, 75 MHz) δ 42.8, 114.3, 120.5, 139.1, 147.1, 150.4, 164.7; HRMS (FAB⁺, m-nitrobenzylalcohol): Calcd for C₇H₈ON₂Cl: 171.0325, Found: 171.0324.

### Example 2

### Preparation of N-2-[2-{4-(2-Methoxyphenyl)-1-piperazinyl}ethyl]amidopyridine (2)

The mixture of 1-(2-methoxy)piperazine (0.20 mL, 1.17 mmol) and K₂CO₃ (0.40 g, 2.93 mmol) in DMF (8 mL) was stirred at 80° C. for 1 h. After cooling down to room temperature, a solution of compound (1) (0.20 g, 1.17 mmol) in DMF (2 mL) and sodium iodide (0.025 g, 0.17 mmol) were added to the mixture. The reaction mixture was stirred at 80° C. for 3 h, cooled to the room temperature. The organic layer was extracted with ethyl acetate, washed with water, and dried over anhydrous MgSO₄. The residue was purified by flash column chromatography (50:1 CH₂Cl₂/MeOH to 20:1 CH₂Cl₂/MeOH) to give product (0.32 g, 84%) as pale yellow oil.

1H NMR (CDCl₃, 300 MHz) 2.82-2.85 (m, 4H), 3.18 (s, 4H), 3.24 (s, 2H), 3.87 (s, 3H), 6.86-7.06 (m, 5H), 7.69-7.75 (m, 1H), 8.25-8.33 (m, 2H), 9.64 (s, 1H); ¹³C NMR (CDCl₃, 75 MHz) δ 50.6, 53.8, 55.4, 62.3, 111.2, 113.9, 118.4, 119.9, 121.0, 123.2, 138.3, 140.9, 148.0, 151.0, 152.3, 169.2; HRMS (FAB⁺, m-nitrobenzylalcohol): Calcd for C₁₈H₂₃O₂N₄: 327.1821, Found: 327.1818.

### Example 3

### Preparation of N-2-[2-{4-(2-Methoxyphenyl)-1-piperazinyl}ethyl]-N-(2-pyridinyl)amine (3)

1 M LiAlH₄/THF (6.70 mL, 6.70 mmol) was slowly added to a solution of compound (2) (0.73 g, 2.24 mmol) in dry THF (10 mL) at 0° C. The mixture was stirred at room temperature under an Ar atmosphere for 3 h. After quenching with saturated aqueous NH₄Cl at 0° C. for 30 min, the mixture was filtrated with ethyl acetate. The organic layer was extracted with ethyl acetate, washed with water, and dried over anhydrous MgSO₄. The residue was purified by flash column chromatography (50:1 CH₂Cl₂/MeOH to 20:1 CH₂Cl₂/MeOH) to give product (0.53 g, 76%) as pale yellow oil.

¹H NMR (CDCl₃, 300 MHz) δ 2.68-2.72 (m, 6H), 3.10 (s, 4H), 3.36-3.41 (m, 2H), 3.87 (s, 3H), 5.14 (s, 1H), 6.40-6.43 (d, 1H, J=8.4 Hz), 6.54-6.59 (m, 1H), 6.85-7.04 (m, 4H), 7.39-7.45 (m, 1H), 8.08-8.11 (m, 1H); ¹³C NMR (CDCl₃, 75 MHz) δ 38.5, 50.7, 53.1, 55.3, 56.8, 107.0, 111.1, 112.7, 118.2, 121.0, 122.9, 137.3, 141.3, 148.2, 152.3, 158.8; HRMS (FAB+, m-nitrobenzylalcohol): Calcd for C₁₈H₂₅ON₄: 313.2028, Found: 313.2030.

### Example 4

### Preparation of trans-N-2-{2-[4-(2-Methoxyphenyl)piperazinyl]ethyl}-N-(2-pyridyl)-N-(4-carboxymethylcyclohexane)carboxamide (4)

Oxalylchloride (0.19 mL, 2.13 mmol) was added to a solution of trans-4-carbomethoxycyclohexane-1-carboxylic acid (0.20 g, 1.05 mmol) in dry CH₂Cl₂ (10 mL), and the mixture was refluxed for 2 h. After removing solvent and unreacted oxalylchloride in vacuo, the product was re-dissolved in dry CH₂Cl₂. Compound (3) (0.22 g, 1.05 mmol) and TEA (0.16 mL, 1.14 mmol) were added to the previous product at 0° C. The reaction mixture was stirred at room temperature under an Ar atmosphere for 2 h. After the mixture was washed with 10% aqueous NaHCO₃ (100 mL), the organic layer was extracted with CH₂Cl₂, and dried over anhydrous MgSO₄. The residue was purified by flash column chromatography (ethyl acetate, 0.1% v/v TEA) to give product (0.28 g, 83%) as pale yellow oil.

¹H NMR (CDCl₃, 300 MHz) δ 1.17-1.25 (m, 2H), 1.62-1.66 (m, 2H), 1.84-1.96 (m, 4H), 2.25-2.29 (m, 2H), 2.58-2.63 (m, 6H), 2.98 (s, 4H), 3.62 (s, 3H), 3.84 (s, 3H), 3.95-4.00 (m, 2H), 6.83-7.00 (m, 4H), 7.24-7.31 (m, 2H), 7.74-7.80 (m, 1H), 8.51-8.53 (m, 1H); ¹³C NMR (CDCl₃, 75 MHz) δ 27.8, 28.3, 41.3, 42.1, 44.8, 50.1, 51.4, 53.0, 55.2, 55.6, 111.0, 117.9, 120.7, 121.9, 122.4, 122.8, 138.2, 140.9, 149.2, 152.0, 175.5, 176.0; HRMS (FAB⁺, m-nitrobenzylalcohol): Calcd for C₂₇H₃₇O₄N₄: 481.2815, Found: 481.2814.

### Example 5

### Preparation of trans-N-2-{2-[4-(2-Methoxyphenyl)piperazinyl]ethyl}-N-(2-pyridyl)-N-(4-hydroxymethylcyclohexane)carboxamide (5)

1 M LiAlH₄/diethyl ether (0.16 mL, 0.16 mmol) was slowly added to the solution of compound (4) (0.076 g, 0.16 mmol) in diethyl ether (5 mL) at 0° C. The reaction mixture was stirred for 30 min at 0° C. under an Ar atmosphere, and quenched with saturated aqueous NH4Cl. The organic layer was extracted with ethyl ether, and then evaporated solvent under reduced pressure. The residue was purified by gravity column chromatography using neutral silica gel (30:1 CH₂Cl₂/MeOH) to give product (0.041 g, 57%) as colorless oil.

¹H NMR (CDCl₃, 300 MHz) δ 1.58-1.85 (m, 10H), 2.59-2.63 (m, 6H), 2.98 (s, 4H), 3.37-3.39 (d, 2H, J=6.0 Hz), 3.84 (s, 3H), 3.96-4.01 (t, 2H, J=6.9 Hz), 6.83-6.98 (m, 4H), 7.22-7.28 (m, 2H), 7.74-7.76 (m, 1H), 8.51-8.53 (m, 1H); ¹³C NMR (CDCl₃, 75 MHz) δ 28.4, 28.9, 39.6, 42.3, 45.2, 50.6, 53.3, 55.3, 56.1, 68.3, 111.1, 118.1, 120.9, 122.3, 122.8, 138.1, 141.3, 149.3, 152.2, 155.8, 176.0; HRMS (FAB⁺, m-nitrobenzylalcohol): Calcd for C₂₆H₃₇O₃N₄: 453.2866, Found: 453.2870.

### Example 6

### Preparation of trans-N-2-{2-[4-(2-Methoxyphenyl)piperazinyl]ethyl}-N-(2-pyridyl)-N-(4-tosyloxymethylcyclohexane)carboxamide (6)

p-Toluenesulfonyl anhydride (0.018 g, 0.056 mmol) and TEA (0.0072 mL, 0.050 mmol) were added to the solution of compound (5) (0.021 g, 0.046 mmol) in CH₂Cl₂ (5 mL). The reaction mixture was stirred at room temperature under an Ar atmosphere for 48 h. After the mixture was washed with 10% aqueous NaHCO₃ (100 mL), the organic layer was extracted with CH₂Cl₂, and dried over anhydrous MgSO₄. The residue was purified by gravity column chromatography using neutral silica gel (20:1 CH₂Cl₂/MeOH) to give product (0.022 g, 75%) as colorless oil. In case of using p-toluenesulfonyl chloride instead of p-Toluenesulfonyl anhydride, the residue was purified by gravity column chromatography using neutral silica gel (20:1 CH₂Cl₂/MeOH) to give product (60%) as colorless oil.

¹H NMR (CDCl₃, 300 MHz) δ 1.25-1.78 (m, 10H), 2.44 (s, 3H), 2.57-2.61 (m, 6H), 2.97 (s, 4H), 3.74-3.76 (d, 2H), 3.84 (s, 3H), 3.96-3.98 (d, 2H), 6.83-6.96 (m, 4H), 7.25-7.33 (m, 4H), 7.72-7.77 (m, 3H), 8.51-8.53 (m, 1H); ¹³C NMR (CDCl₃, 75 MHz) δ 21.6, 28.0, 28.5, 36.4, 41.8, 45.3, 50.6, 53.4, 55.3, 56.1, 74.9, 111.1, 118.1, 120.9, 122.2, 122.4, 122.8, 127.8, 129.8, 132.9, 138.2, 141.3, 144.7, 149.3, 152.2, 155.8, 175.6; HRMS (FAB⁺, m-nitrobenzylalcohol): Calcd for C₃₃H₄₃O₅N₄S: 607.2954, Found: 607.2954.

### Example 7

### Preparation of ¹⁸F-Mefway

¹⁸F-fluoride was produced in the Eclipse HP cyclotron (Siemens) using oxygen-18 enriched water (¹⁸O to ¹⁸F using p, n reaction). Fluorine-18 radioactivity was counted in a Capintec dose calibrator The mefway precursor molecule, tosylate was reacted with ¹⁸F-fluoride at 110° C. for 15 min in hot cell.n t. The crude product was purified in a reverse-phase HPLC C₁₈ Econosil column 250.times.10 mm (Alltech Assoc. Inc.) with 45% acetonitrile:55% water containing 0.1% triethylamine with a flow rate of 5 mL/min. The retention time of trans ¹⁸F-mefway was expected to be approx. 12 minutes. The radiosynthesis and purification was complete in 1.5 hrs.

### Comparative Example 1

### Preparation of trans-N-2-{2-[4-(2-Methoxyphenyl)piperazinyl]ethyl}-N-(2-pyridyl)-N-(4carboxymethylcyclohexane)carboxamide (4) by using BOP

Trans-N-2-{2-[4-(2-Methoxyphenyl)piperazinyl]ethyl}-N-(2-pyridyl)-N-(4carboxymethylcyclohexane)carboxamide (4) was prepared in the same manner as in Example 4, except that benzotriazole-1-yl-oxy-tris-(dimethylamino)-phosphonium hexafluorophosphate (BOP) used instead of oxalyl chloride. The residue was purified by flash column chromatography (ethyl acetate, 0.1% v/v TEA) to give product (less than 10%) as pale yellow oil.

Comparative example 2: Preparation of trans-N-2-{2-[4-(2-Methoxyphenyl)piperazinyl]ethyl}-N-(2-pyridyl)-N-(4-hydroxymethylcyclohexane)carboxamide (5) by using THF

Trans-N-2-{2-[4-(2-Methoxyphenyl)piperazinyl]ethyl}-N-(2-pyridyl)-N-(4-hydroxymethylcyclohexane)carboxamide (5) was prepared in the same manner as in Example 5, except that THF used instead of diethyl ether. In this case, a selective reduction of the carbonyl group was impossible or the residue was purified by gravity column chromatography using neutral silica gel (30:1 CH₂Cl₂/MeOH) to give product (less than 30%) as colorless oil.

### Comparative Example 3

### Preparation of trans-N-2-{2-[4-(2-Methoxyphenyl)piperazinyl]ethyl}-N-(2-pyridyl)-N-(4-hydroxymethylcyclohexane)carboxamide (5) by using other reducing agents

Trans-N-2-{2-[4-(2-Methoxyphenyl)piperazinyl]ethyl}-N-(2-pyridyl)-N-(4-hydroxymethylcyclohexane)carboxamide (5) was prepared in the same manner as in Example 5, except that NaBH₄, Ca(BH₄)₂ or diisobutylaluminumhydride used instead of LAH. In this case, a selective reduction of the carbonyl group was impossible.

What is claimed is:

1. A process for preparing a compound of the formula 6 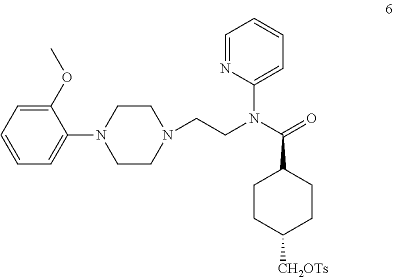
which process comprises the steps of:
a) adding oxalyl chloride to 4-carbomethoxycyclohexane-1-carboxylic acid to form a reaction mixture and reacting the reaction mixture with a compound of the formula 3 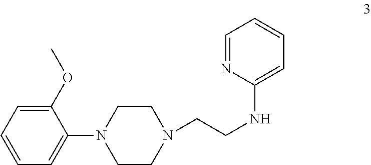
to form a compound of the formula 4 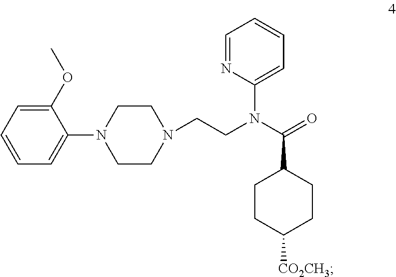
b) reduction of the compound of the formula 4 with LiAlH₄ in diethyl ether to form a compound of the formula 5 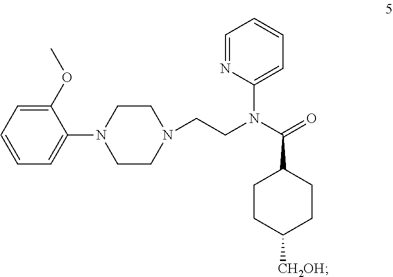
and
c) tosylating the compound of the formula 5 with p-toluenesulfonyl anhydride or p-toluenesulfonyl chloride to form the compound of the formula 6.

2. The process according to claim 1, wherein the adding oxalyl chloride to 4-carbomethoxycyclohexane-1-carboxylic acid is in CH₂Cl₂.

3. The process according to claim 1, wherein the process further comprises the step of:
reacting 2-aminopyridine with chloroacetyl chloride and then adding a reducing agent to form the compound of the formula 3.

4. The process according to claim 3, wherein the reducing agent is LiAlH₄, NaBH₄, Ca(BH₄)₂, diisobutylaluminumhydride, AlH₃ or sodium bis(2-methoxy-ethoxy)aluminum hydride.

5. A process for preparing a compound of the formula 5 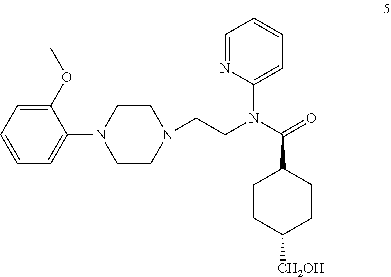
which process comprises the steps of:
a) adding oxalyl chloride to 4-carbomethoxycyclohexane-1-carboxylic acid to form a reaction mixture and reacting the reaction mixture with a compound of the formula 3 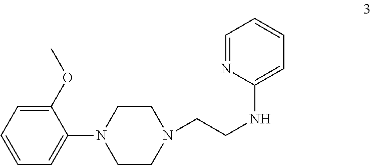
to form a compound of the formula 4 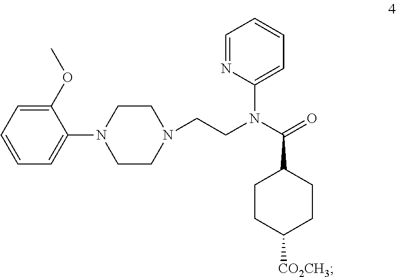
and
b) reduction of the compound of the formula 4 with LiAlH₄ in diethyl ether to form the compound of the formula 5.

6. The process according to claim 5, wherein the adding oxalyl chloride to 4-carbomethoxycyclohexane-1-carboxylic acid is in CH₂Cl₂.

7. The process according to claim 5, wherein the process further comprises the step of:
reacting 2-aminopyridine with chloroacetyl chloride and then adding a reducing agent to form the compound of the formula 3.

8. The process according to claim 7, wherein the reducing agent is LiAlH₄, NaBH₄, Ca(BH₄)₂, diisobutylaluminumhydride, AlH₃ or sodium bis(2-methoxy-ethoxy)aluminum hydride.

9. A process for preparing ¹⁸F-mefway, which process comprises the steps of:
a) adding oxalyl chloride to 4-carbomethoxycyclohexane-1-carboxylic acid to form a reaction mixture and reacting the reaction mixture with a compound of the formula 3 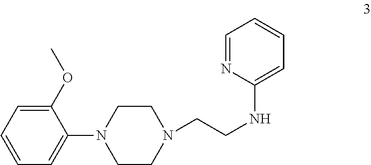
to form a compound of the formula 4 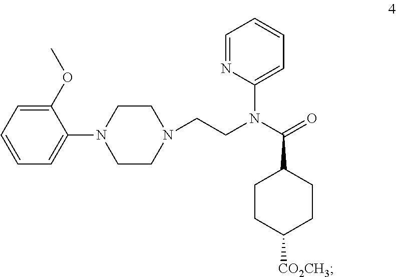
b) reduction of the compound of the formula 4 with LiAlH₄ in diethyl ether to form a compound of the formula 5 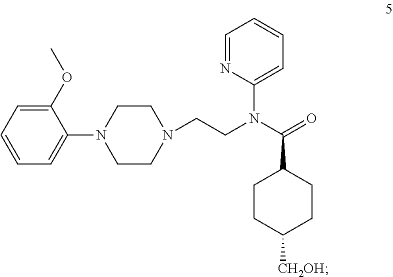
c) tosylating the compound of the formula 5 with p-toluenesulfonyl anhydride or p-toluenesulfonyl chloride to form a compound of the formula 6 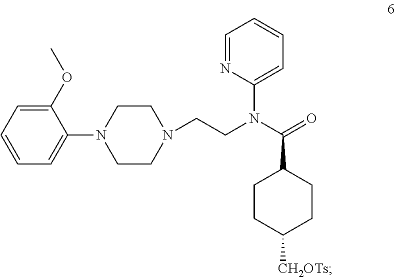
and
d) reacting the compound of the formula 6 with ¹⁸F-fluoride to form ¹⁸F-mefway.

10. The process according to claim 9, wherein the adding oxalyl chloride to 4-carbomethoxycyclohexane-1-carboxylic acid is in CH₂Cl₂.

11. The process according to claim 9, wherein the process further comprises the step of:
reacting 2-aminopyridine with chloroacetyl chloride and then adding a reducing agent to form the compound of the formula 3.

12. The process according to claim 11, wherein the reducing agent is LiAlH₄, NaBH₄, Ca(BH₄)₂, diisobutylaluminumhydride, AlH₃ or sodium bis(2-methoxy-ethoxy)aluminum hydride.
